(19) United States
(12) Patent Application Publication
(10) Pub. No.: US 2026/0060657 A1
(43) Pub. Date: Mar. 5, 2026

(54) ULTRASOUND DIAGNOSIS APPARATUS, ULTRASOUND DIAGNOSIS METHOD, AND NON-TRANSITORY COMPUTER READABLE STORAGE MEDIUM

(71) Applicant: Canon Medical Systems Corporation, Otawara-shi (JP)

(72) Inventors: Ryosuke IWASAKI, Tokyo (JP); Tetsuya YOSHIDA, Tokyo (JP); Hayato IKEDA, Kawasaki (JP)

(73) Assignee: Canon Medical Systems Corporation, Otawara-shi (JP)

(21) Appl. No.: 19/309,837

(22) Filed: Aug. 26, 2025

(30) Foreign Application Priority Data
Aug. 28, 2024 (JP) 2024-146569

(51) Int. Cl. A61B 8/00

(52) U.S. Cl. CPC A61B 8/5253; A61B 8/4477; A61B 8/463; A61B 8/488; A61B 8/5207; A61B 8/5269; A61B 8/56

(57) ABSTRACT

According to one embodiment, an ultrasound diagnosis apparatus includes processing circuitry. The processing circuitry is configured to acquire a first echo signal based on a first ultrasound beam and a second echo signal based on a second ultrasound beam, the second ultrasound beam covering an area at least partially overlapping, and differing in an elevation direction from, an area covered by the first ultrasound beam. The processing circuitry is configured to calculate a differential signal representing a difference between the first echo signal and the second echo signal. The processing circuitry is configured to display a differential image based on the differential signal.

### CROSS-REFERENCE TO RELATED APPLICATION

This application is based upon and claims the benefit of priority from Japanese Patent Application No. 2024-146569, filed Aug. 28, 2024, the entire contents of which are incorporated herein by reference.

### FIELD

Embodiments described herein relate generally to an ultrasound diagnosis apparatus, an ultrasound diagnosis method, and a non-transitory computer readable storage medium.

### BACKGROUND

In a needle biopsy or radiofrequency ablation (RFA) treatment, there is a case where a procedure is performed while checking the positional relationship between a target and a needle tip of a puncture needle with the guide of ultrasound imaging. At this time, there is a means for visually enhancing the needle portion depicted in an ultrasound image.

In general, in the course of performing such a procedure, there is a case where the position of the needle tip appears to be located within the target, but is actually shifted forward or backward of the target, since the ultrasound image being displayed in real time is a two-dimensional cross-sectional image. In this case, since the shifted needle tip not depicted in the ultrasound image cannot be visually enhanced, it is difficult for the operator to grasp the position gap of the needle tip.

Such difficulty occurs not only for a needle tip of a puncture needle, but also for other detection targets of which there is a demand for checking, such as a catheter and a stent, in an ultrasound-guided procedure.

### BRIEF DESCRIPTION OF THE DRAWINGS

FIG. 1 is a block diagram showing a configuration example of an ultrasound diagnosis apparatus according to the present embodiment.

FIG. 2 is a flowchart showing an operation example of an ultrasound diagnosis apparatus according to a first embodiment.

FIG. 3 is a diagram showing a first generation example of an ultrasound beam.

FIG. 4 is a diagram showing beam setting for a detection target according to the first generation example.

FIG. 5 is a diagram showing a display example of an ultrasound image and a superimposition image according to the first generation example.

FIG. 6 is a diagram showing a second generation example of an ultrasound beam.

FIG. 7 is a flowchart showing an operation example of an ultrasound diagnosis apparatus according to a second embodiment.

FIG. 8 is a diagram showing a third generation example of an ultrasound beam.

FIG. 9 is a diagram showing a fourth generation example of an ultrasound beam.

FIG. 10 is a diagram showing beam setting for a detection target according to the fourth generation example.

FIG. 11 is a diagram showing a display example of an ultrasound image and a superimposition image according to the fourth generation example.

FIG. 12 is a diagram showing a fifth generation example of an ultrasound beam.

FIG. 13 is a diagram showing beam setting for a detection target according to the fifth generation example.

FIG. 14 is a diagram showing a display example of an ultrasound image and a superimposition image according to the fifth generation example.

FIG. 15 is a diagram showing a display example of an ultrasound image and a superimposition image in a case where an ROI is a target.

### DETAILED DESCRIPTION

In general, according to one embodiment, an ultrasound diagnosis apparatus includes processing circuitry. The processing circuitry is configured to acquire a first echo signal based on a first ultrasound beam and a second echo signal based on a second ultrasound beam, the second ultrasound beam covering an area at least partially overlapping, and differing in an elevation direction from, an area covered by the first ultrasound beam. The processing circuitry is configured to calculate a differential signal representing a difference between the first echo signal and the second echo signal. The processing circuitry is configured to display a differential image based on the differential signal.

Hereinafter, an ultrasound diagnosis apparatus, an ultrasound diagnosis method, and a non-transitory computer-readable storage medium according to the present embodiment will be described with reference to the accompanying drawings. In the embodiments to be described below, portions denoted by the same reference numerals are assumed to perform similar operations, and repetitive descriptions will be suitably omitted.

### First Embodiment

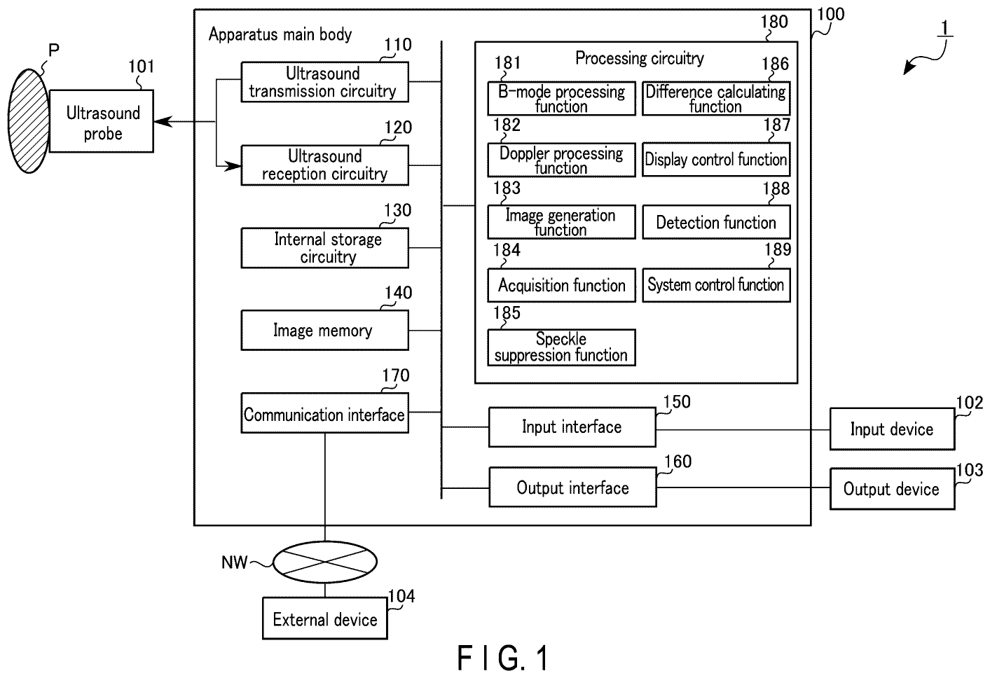

FIG. 1 is a diagram showing a configuration example of an ultrasound diagnosis apparatus according to the present embodiment. The ultrasound diagnosis apparatus 1 shown in FIG. 1 includes an apparatus main body 100 and an ultrasound probe 101. The apparatus main body 100 is connected to an input device 102 and an output device 103. Also, the apparatus main body 100 is connected to an external device 104 via a network NW. The external device 104 is, for example, a server equipped with a picture archiving and communication system (PACS) and a workstation capable of executing post-processing.

The ultrasound probe 101 is configured to execute, for example, ultrasound scanning of a scan region in a living body P, which is a subject, under the control of the apparatus main body 100. The ultrasound probe 101 is, for example, an acoustic lens, one or more matching layers, a plurality of transducers (piezoelectric elements), a backing material, etc. The acoustic lens is formed of, for example, silicon rubber, and is configured to converge an ultrasound beam. The one or more matching layers perform impedance matching between the plurality of transducers and a living body. The backing material prevents propagation of ultrasound waves from the plurality of transducers backward with respect to the radiation direction. The ultrasound probe 101 is, for example, a linear probe, a convex probe, or the like. The ultrasound probe 101 is detachably connected to the apparatus main body 100. A button to be pressed in the event of an offset process or an operation (freeze operation) to freeze the ultrasound images may be disposed in the ultrasound probe 101.

The plurality of transducers generate an ultrasound wave based on a drive signal supplied from ultrasound transmission circuitry 110 (to be described later) of the apparatus main body 100. Thereby, an ultrasound wave is transmitted from the ultrasound probe 101 to the living body P. The ultrasound wave transmitted from the ultrasound probe 101 to the living body P is sequentially reflected on a surface of acoustic impedance discontinuity at a body tissue of the living body P, and is received by a plurality of piezoelectric transducers as an echo signal. The amplitude of the received echo signal depends on the difference in acoustic impedance on the surface of discontinuity on which the ultrasound wave is reflected. If a transmitted ultrasound pulse is reflected on a surface of a moving blood flow, a cardiac wall, or the like, the echo signal is, due to the Doppler effect, subject to a frequency shift, depending on a velocity component of the moving body in the ultrasound transmission direction. The ultrasound probe 101 is configured to receive an echo signal from the living body P and convert it into an electric signal.

FIG. 1 shows, as an example, a connection relationship between a single ultrasound probe 101 and the apparatus main body 100. However, a plurality of ultrasound probes 101 may be connected to the apparatus main body 100. Which of the connected ultrasound probes 101 is to be used for ultrasound scanning can be freely selected, for example, via a software button on a touch panel, to be described later.

The apparatus main body 100 is an apparatus configured to generate an ultrasound image based on an echo signal received by the ultrasound probe 101 (simply referred to as an “echo signal”). The apparatus main body 100 includes ultrasound transmission circuitry 110, ultrasound reception circuitry 120, internal storage circuitry 130, an image memory 140, an input interface 150, an output interface 160, a communication interface 170, and processing circuitry 180.

The ultrasound transmission circuitry 110 is a processor configured to supply a drive signal to the ultrasound probe 101. The ultrasound transmission circuitry 110 is realized by, for example, a trigger generation circuit, a delay circuit, a pulser circuit, or the like. The trigger generation circuit is configured to repeatedly generate a rate pulse for forming transmission ultrasound at a predetermined rate frequency. The delay circuit is configured to provide each rate pulse generated by the trigger generation circuit with a delay time required for each of the piezoelectric transducers to render an ultrasound wave generated by the ultrasound probe into a converging beam and determine a transmission directivity. The pulser circuit is configured to apply a drive signal (drive pulse) to the plurality of ultrasound transducers provided in the ultrasound probe 101 at a timing based on the rate pulse. By varying the delay time to be provided to each rate pulse by the delay circuit, it is possible to freely adjust the transmission direction from the surfaces of the piezoelectric transducers.

Also, the ultrasound transmission circuitry 110 is capable of freely varying the output intensity of the ultrasound with the drive signal. By increasing the output intensity of the ultrasound diagnosis apparatus, it is possible to obtain an echo signal with a high signal-to-noise ratio (SNR) at the time of reception.

In general, if an ultrasound wave is propagated through a living body P, the intensity of ultrasound vibration (also referred to as “acoustic power”) corresponding to the output intensity is attenuated. The attenuation of the acoustic power is caused by absorption, scattering, reflection, etc. The degree of decrease in acoustic power depends on the frequency and the radiation-direction distance of the ultrasound wave. For example, the greater the frequency of the ultrasound wave, the higher the degree of attenuation. Also, the longer the radial-direction distance of the ultrasound wave, the higher the degree of attenuation.

The ultrasound reception circuitry 120 is a processor configured to subject the echo signal received by the ultrasound probe 101 to various processes, and to generate a reception signal. The ultrasound reception circuitry 120 generates a reception signal for an ultrasound echo signal obtained by the ultrasound probe 101. Specifically, the ultrasound reception circuitry 120 is realized by, for example, a preamplifier, an A/D converter, a demodulator, a beam former (adder), and the like. The preamplifier is configured to perform a gain correction process by amplifying the echo signal received by the ultrasound probe 101 for each channel. The A/D converter is configured to convert the gain-corrected echo signal into a digital signal. The demodulator is configured to demodulate the digital signal. The beam former is configured, for example, to provide a delay time required for determining a reception directivity to each of the demodulated digital signals and to sum the digital signals to which the delay time has been provided. As a result of the summing by the beam former, a reception signal with an emphasis on reflection components from a direction corresponding to the reception directivity is generated. The reception signal may also be referred to as an “IQ signal”. The ultrasound reception circuitry 120 may also be configured to allow the internal storage circuitry 130 to store the reception signal (IQ signal), or to output the reception signal (IQ signal) to the external device 104 via the communication interface 170.

The internal storage circuitry 130 includes, for example, a processor-readable storage medium such as a magnetic storage medium, an optical storage medium, or a semiconductor memory. The internal storage circuitry 130 is configured to store programs and various types of data for realizing ultrasound transmission and reception. Such programs and various types of data may be stored in advance in, for example, the internal storage circuitry 130. Moreover, such programs and various types of data may be stored in a non-transitory storage medium and distributed, read from the non-transitory storage medium, and installed onto the internal storage circuitry 130. Also, the internal storage circuitry 130 is configured to store B-mode image data, contrast-enhanced image data, and blood-flow images generated by the processing circuitry 180, in accordance with an operation input via the input interface 150. The internal storage circuitry 130 may also be configured to transfer the stored image data to the external device 104 or the like via the communication interface 170. Note that the internal storage circuitry 130 may be configured to store the reception signal (IQ signal) generated by the ultrasound reception circuitry 120, or to transfer the reception signal (IQ signal) to the external device 104 via the communication interface 170.

The internal storage circuitry 130 may be a drive device, etc. configured to read and write a variety of information to and from a portable storage medium such as a CD drive, a DVD drive, a flash memory, or the like. The internal storage circuitry 130 may also be configured to write the stored data into a portable storage medium, and to allow the external device 104 to store the data via the portable storage medium.

The image memory 140 is, for example, a processor-readable storage medium such as a magnetic storage medium, an optical storage medium, or a semiconductor memory. The image memory 140 stores image data corresponding to a plurality of frames immediately before a freeze operation input via the input interface 150. The image data stored in the image memory 140 is, for example, displayed in a continuous mode (cine mode).

The internal storage circuitry 130 and the image memory 140 are not necessarily realized by independent storage devices. The internal storage circuitry 130 and the image memory 140 may be realized by a single storage device. Also, the above-described internal storage circuitry 130 and the image memory 140 may be respectively realized by a plurality of storage devices.

The input interface 150 receives various instructions from the operator via the input device 102. The input device 102 is, for example, a mouse, a keyboard, a panel switch, a slider switch, a trackball, a rotary encoder, an operation panel, or a touch command screen (TCS). The input interface 150 is connected to the processing circuitry 180 via a bus, converts an operation instruction input by the operator into an electric signal, and outputs the electric signal to the processing circuitry 180. The input interface 150 is not limited to a configuration that is connected to physical operation components such as a mouse and a keyboard. Examples of the input interface include circuitry configured to receive an electric signal corresponding to an operation instruction input from an external input device provided separately from the ultrasound diagnosis apparatus 1, and to output the received electric signal to the processing circuitry 180.

The output interface 160 is, for example, an interface for outputting an electric signal from the processing circuitry 180 to the output device 103. The output device 103 is a given display, such as a liquid crystal display, an organic EL display, an LED display, a plasma display, a CRT display, or the like. The output device 103 may be a touch-panel display that also functions as the input device 102. The output device 103 may further include a speaker for outputting speech, in addition to a display. The output interface 160 is, for example, connected to the processing circuitry 180 via a bus, and configured to output an electric signal from the processing circuitry 180 to the output device 103.

The communication interface 170 is, for example, an interface connected to the external device 104 via the network NW and configured to perform data communication with the external device 104.

The processing circuitry 180 is, for example, a processor that functions as a central nerve of the ultrasound diagnosis apparatus 1. The processing circuitry 180 is configured to execute programs stored in the internal storage circuitry 130, thereby realizing functions corresponding to the respective programs. The processing circuitry 180 includes, for example, a B-mode processing function 181, a Doppler processing function 182, an image generation function 183, an acquisition function 184, speckle suppression function 185, a difference calculating function 186, a display control function 187, a detection function 188, and a system control function 189.

The B-mode processing function 181 is a function of generating B-mode data based on a reception signal received from the ultrasound reception circuitry 120. With the B-mode processing function 181, the processing circuitry 180 subjects, for example, the reception signal received from the ultrasound reception circuitry 120 to an envelope detection process, a logarithmic compression process, and the like, and generates data (B-mode data) in which a signal intensity is expressed by a degree of brightness. The generated B-mode data is stored in an unillustrated RAW data memory as B-mode RAW data on two-dimensional ultrasound scanning lines (raster).

The Doppler processing function 182 is a function of frequency-analyzing the reception signal received from the ultrasound reception circuitry 120, thereby generating data (Doppler information) in which motion information based on the Doppler effect of a moving body in a region of interest (ROI) set in a scan region is extracted. The generated Doppler information is stored in an unillustrated RAW data memory as Doppler RAW data on two-dimensional ultrasound scanning lines (also referred to as “Doppler data”).

Specifically, the processing circuitry 180 generates, with the Doppler processing function 182, Doppler data indicating estimated motion information by estimating a velocity, a direction, their distribution, etc., at a plurality of sample points as motion information of a moving body. The moving body is, for example, a blood flow, tissues such as a heart wall, a contrast agent, or the like. The processing circuitry 180 generates, with the Doppler processing function 182, Doppler data indicating estimated blood flow information by estimating an average blood flow velocity, a blood flow velocity variance, a blood flow signal power value, etc. at a plurality of sample points as motion information of the blood flow (blood flow information).

Moreover, the processing circuitry 180 may execute, with the Doppler processing function 182, a color Doppler technique known as color flow mapping (CFM). In the CFM technique, ultrasound transmission and reception are performed multiple times on a plurality of scanning lines. In the CFM technique, for example, signals calculated from still or slow-moving tissues (clutter signals) are suppressed by applying a moving target indicator (MTI) filter to rows of data at the same position, thereby extracting a signal calculated from a blood flow. In the CFM technique, blood flow information such as the velocity, variance, power, etc. of the blood flow is estimated using the extracted blood flow signal. With the image generation function 183 to be discussed below, a distribution of the estimated blood flow information is generated as, for example, ultrasound image data (color Doppler image data) in a two-dimensional color representation. Hereinafter, a mode of an ultrasound diagnosis apparatus using the color Doppler technique will be referred to as a “blood flow imaging mode”. It is assumed that the color representation refers to representation of a distribution of the blood flow information in association with a predetermined color code, and encompasses a grayscale representation.

The blood flow imaging mode includes various types from which selection can be made according to the desired clinical information. In general, a velocity-display blood flow imaging mode for allowing the direction and the average velocity of the blood flow to be visually recognizable, and the power-display blood flow imaging mode for allowing the power of the blood flow signal to be visually recognizable, are known.

The velocity-display blood flow imaging mode is a mode for displaying the color corresponding to the Doppler shift frequency according to the direction and the average velocity of the blood flow. In the velocity-display blood flow imaging mode, the difference in velocity between flows in both directions is represented as a difference in hue, with the incoming flow expressed in a red hue, and the outgoing flow expressed in a blue hue. The velocity-display blood flow imaging mode is also referred to as a color Doppler mode or a color Doppler imaging (CDI) mode.

The power-display blood flow imaging mode is, for example, a mode in which the power of a blood flow signal is expressed as a change in hues, brightness (luminance), or colorfulness. The power-display blood flow imaging mode is also referred to as a power Doppler (PD) mode. The power-display blood flow imaging mode may also be referred to as a “high-sensitivity blood flow imaging mode”, since the blood flow can be depicted with high sensitivity as compared to the velocity-display blood flow imaging mode.

The image generation function 183 is a function of generating B-mode image data based on data generated with the B-mode processing function 181. With the image generation function 183, the processing circuitry 180 converts (scan-coverts), for example, ultrasound scan line signals into video-format scan line signals (as in television), thus generating image data for display (display image data). Specifically, the processing circuitry 180 performs RAW-pixel conversion on B-mode RAW data stored in the RAW data memory, such as coordinate conversion corresponding to the format of ultrasound scanning by the ultrasound probe 101, thereby generating two-dimensional B-mode image data (also referred to as an “ultrasound image data”) configured of pixels. In other words, the processing circuitry 180 generates, with the image generation function 183, a plurality of ultrasound images (medical images) respectively corresponding to a plurality of consecutive frames through ultrasound transmission and reception.

The image generation function 183 also includes a function of generating Doppler image data based on data generated with the Doppler processing function 182. For example, the image generation function 183 executes RAW-pixel conversion on Doppler RAW data stored in the RAW data memory, thereby generating Doppler image data in which the blood flow information is visualized. Doppler image data is average-velocity image data, variance image data, power image data, or image data of a combination thereof. The processing circuitry 180 generates, as the Doppler image data, color Doppler image data in which the blood flow information is represented by colors, and Doppler image data in which a single piece of blood flow information is represented as a grayscale waveform. The color Doppler image data is generated prior to execution of the blood flow imaging mode.

The acquisition function 184 acquires a first echo signal based on a first ultrasound beam, and acquires a second echo signal based on a second ultrasound beam covering an area at least partially overlapping, and differing in an elevation direction from, an area covered by the first ultrasound beam.

The speckle suppression function 185 executes a speckle suppression process on the first echo signal and the second echo signal.

The difference calculating function 186 calculates a differential signal representing a difference between the first echo signal and the second echo signal.

The display control function 187 displays a differential image based on the differential signal.

Also, the display control function 187 is a function of displaying an image generated based on various types of ultrasound image data with the image generation function 183 on a display that functions as the output device 103. Specifically, with the display control function 187, the processing circuitry 180 controls, for example, the displaying, on a display, of images based on the B-mode image data generated with the image generation function 183 or the Doppler image data, or image data containing both of them.

More specifically, with the display control function 187, the processing circuitry 180 converts (scan-coverts), for example, ultrasound scan line signals into video-format scan line signals (as in television), thus generating display image data. Moreover, the processing circuitry 180 may execute, on the display image data, various processes such as dynamic range correction, brightness correction, contrast correction, gamma curve correction, and RGB conversion. Furthermore, the processing circuitry 180 may add incidental information such as character information of various parameters, gradations, body marks, etc. on the display image data. The processing circuitry 180 may be configured to generate and cause a display to display a graphical user interface (GUI) for allowing the operator to input various instructions via an input device.

The detection function 188 detects whether or not a detection target is depicted in the differential image. Also, the detection function 188 determines, if the detection target is depicted in the differential image, that at least a portion of the detection target is located outside of a plane of a desired cross-sectional image. It is assumed in the present embodiment that the detection target is a puncture needle, in particular, a needle tip of a puncture needle; however, the detection target is not limited thereto, and is applicable to any target of which there is a demand for checking the location, such as a catheter and a stent, in an ultrasound-guided procedure.

The system control function 189 is a function of collectively controlling the entire operation of the ultrasound diagnosis apparatus 1.

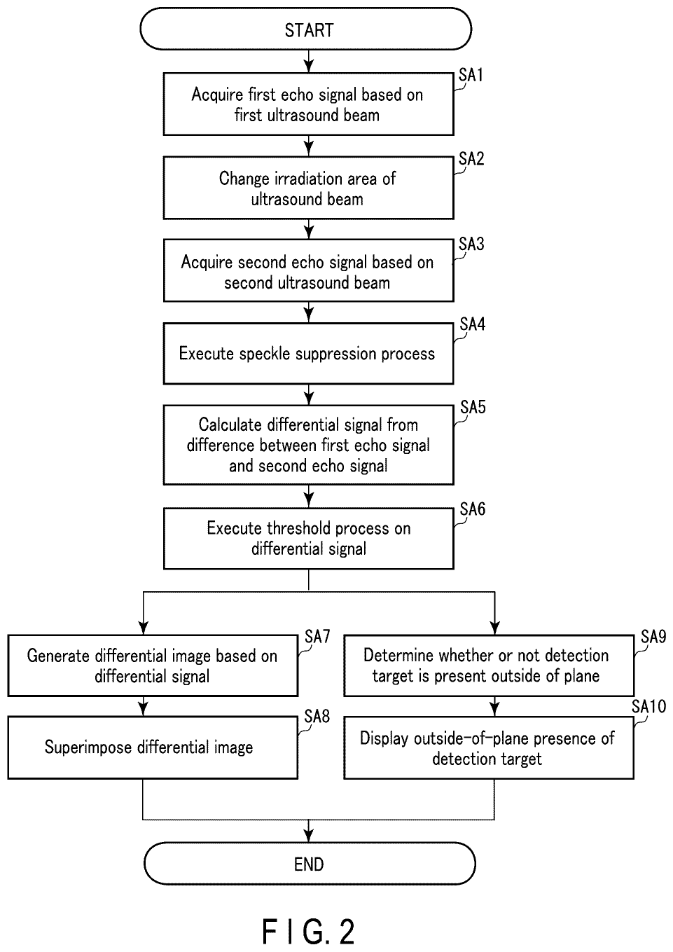

Next, an operation example of the ultrasound diagnosis apparatus 1 according to the first embodiment will be described with reference to the flowchart shown in FIG. 2.

At step SA1, the processing circuitry 180 acquires, with the acquisition function 184, a first echo signal based on a first ultrasound beam. Specifically, the ultrasound transmission circuitry 110, the ultrasound probe 101, and the ultrasound reception circuitry 120 are controlled to transmit, to the living body P, an ultrasound beam set to a desired beam thickness for imaging a region of interest (ROI), thereby acquiring a first echo signal based on a first ultrasound beam.

At step SA2, with the acquisition function 184, the processing circuitry 180 performs setting to change a beam irradiation area to be different from an irradiation area of the first ultrasound beam in an elevation direction. Specifically, the ultrasound transmission circuitry 110, the ultrasound probe 101, and the ultrasound reception circuitry 120 are controlled, and an area of a second ultrasound beam, to be described later, is set.

At step SA3, the processing circuitry 180 acquires, with the acquisition function 184, a second echo signal based on the second ultrasound beam, in accordance with the condition changed at step SA2. Specifically, similarly to step SA1, the ultrasound transmission circuitry 110, the ultrasound probe 101, and the ultrasound reception circuitry 120 are controlled, thereby acquiring a second echo signal based on a second ultrasound beam.

At step SA4, the processing circuitry 180 executes, with the speckle suppression function 185, a speckle suppression process on the first echo signal and the second echo signal. Since the speckle pattern fluctuates according to a change in the beam spatial area in the elevation direction, there may be a possibility that a portion other than the detection target may represent a high signal after the differential processing, possibly resulting in error detection. Accordingly, a speckle suppression process is performed to prevent a portion other than the detection target from representing a high signal. Specifically, the speckle suppression process is performed by, for example, a filter process such as spatial low-pass filtering, repeated averaging of echo signals, or a combination thereof.

At step SA5, the processing circuitry 180 calculates, with the difference calculating function 186, a difference between the first echo signal and the second echo signal subjected to the speckle suppression process, and calculates a differential signal. The difference may be calculated by subtracting the first echo signal from the second echo signal, or subtracting the second echo signal from the first echo signal.

At step SA6, the processing circuitry 180 executes, with the difference calculating function 186, a threshold process on the differential signal to extract a signal value equal to or greater than a threshold value. The threshold process is a process for removing a non-detection target. That is, for the desire to detect a detection target that is not present in the spatial area of the first ultrasound beam but is present in the spatial area of the second ultrasound beam, it suffices that a threshold process is executed for the differential signal, since a signal value that becomes negative if the first echo signal is subtracted from the second echo signal is not considered to represent a detection target. If the second echo signal is subtracted from the first echo signal, a signal value that becomes positive through the subtraction can be determined as not representing a detection target.

At step SA7, the processing circuitry 180 generates, with the display control function 187, a differential image based on the differential signal, and displays, with the display control function 187, a differential image on an external display, or the like.

At step SA8, the processing circuitry 180 superimposes, with the display control function 187, the differential image on a first ultrasound image based on the first echo signal (hereinafter referred to as a “superimposition image”). The superimposing may be performed by making the display colors of the differential image and the first ultrasound image different. Moreover, the differential image may be displayed alone.

At step SA9, based on the differential signal, the processing circuitry 180 determines, with the detection function 188, whether or not the detection target is present outside of the plane of a desired cross-sectional image. If, for example, a signal value equal to or greater than a threshold value does not exist in the differential signal, it can be considered that the detection target is not represented by the differential signal, and it can be determined that the detection target is not present outside of the plane of a desired cross-sectional image, that is, the detection target is present within the cross-sectional image.

At step SA10, the processing circuitry 180 may cause a display to display, with the display control function 187, a message indicating that the detection target is present outside of the plane, together with a differential image or a superimposition image. Moreover, the processing circuitry 180 may be configured to cause a speaker to output speech or an alert sound indicating that the detection target is present outside of the plane.

Instead of executing the speckle suppression process at step SA4 and the threshold process at step SA6, a differential image may be generated.

Moreover, steps SA7 and SA8 and steps SA9 and SA10 may be independently processed, or successively processed. That is, after the processing at steps SA7 and SA8 are performed, the processing at steps SA9 and SA10 may be skipped, in such a manner, for example, that only a superimposition image is displayed on a display. Alternatively, the processing at steps SA9 and SA10 may be performed after skipping steps SA7 and SA8, in such a manner, for example, that a message indicating whether or not the detection target is present outside of the plane is displayed while an ultrasound image based on the first echo signal is being displayed on a display.

If, for example, steps SA7 to SA10 are successively performed, the determination process at step SA9, for example, may be executed based on the differential image. Specifically, the processing circuitry 180 may determine, with the detection function 188, that the detection target is present outside of the plane if a portion with a signal value equal to or greater than a threshold value is present in the differential image so as to be continuous from a desired cross-sectional image in the differential image. On the other hand, if a portion with a signal value equal to or greater than a threshold value is not present in the differential image, the processing circuitry 180 may determine that the detection target is not present outside of the plane.

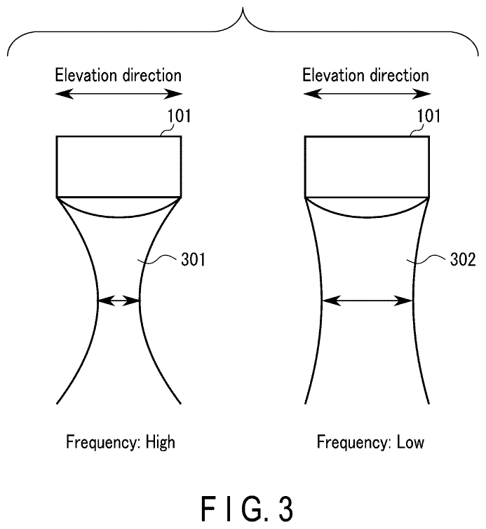

Next, a first generation example of an ultrasound beam according to the first embodiment will be described with reference to FIG. 3.

In the first generation example shown in FIG. 3, an ultrasound beam transmitted from the ultrasound probe 101 or an ultrasound beam received by the ultrasound probe 101 have different frequencies. A first ultrasound beam 301 is transmitted or received using a first frequency having a desired beam thickness in the elevation direction, thereby obtaining a first echo signal. Thereafter, a second ultrasound beam 302 is transmitted or received using a second frequency having a frequency lower than the first frequency, thereby acquiring a second echo signal. An ultrasound beam can be generated in such a manner that the higher the transmission frequency, the smaller the focus becomes, that is, the smaller the beam thickness becomes. In other words, the first ultrasound beam 301 has a higher frequency, and has a smaller beam thickness. On the other hand, the second ultrasound beam 302 has a lower frequency, and has a greater beam thickness.

Of course, the frequency of the ultrasound beam may be set both in transmission and reception of an ultrasound beam. That is, the first echo signal may be acquired by transmitting and receiving the first ultrasound beam 301 using the first frequency, and the second echo signal may be acquired by transmitting and receiving the second ultrasound beam 302 using the second frequency. Thereby, the second echo signal includes part of the first echo signal, and includes, in the first generation example, the entire first echo signal, and is thus capable of acquiring a signal outside of the area of the first ultrasound beam 301.

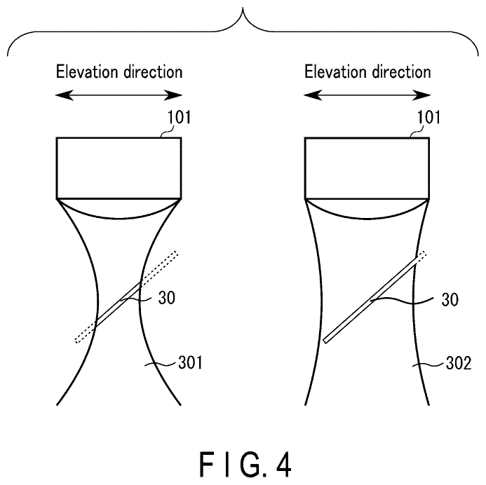

Next, beam setting for the detection target according to the first generation example will be described with reference to FIG. 4.

In the example shown in FIG. 4, it is assumed that the detection target is a puncture needle 30, and that an echo signal is acquired based on the first ultrasound beam 301 and the second ultrasound beam 302, similarly to FIG. 3. The portion in which the puncture needle 30 is depicted in the first ultrasound beam 301 is shown by the solid line, and the portion in which the puncture needle 30 is not depicted in the first ultrasound beam 301 is shown by the dashed line. Since the puncture needle 30 is partially located outside of the area of the first ultrasound beam 301, it is difficult to determine, only from the ultrasound image based on the first ultrasound beam 301, whether or not the puncture needle 30 is present outside of the plane. On the other hand, since the puncture needle 30 is included in the area of the second ultrasound beam 302 and is depicted in the ultrasound image based on the second ultrasound beam 302, it is possible to determine that the puncture needle 30 is present outside of the plane of the ultrasound image based on the first ultrasound beam 301.

By thus varying the frequency of the ultrasound beam, it is possible with the second ultrasound beam 302 to acquire echo signals from different areas, thus acquiring an echo signal in a spatial area outside of the plane of the first ultrasound beam 301. It is thus possible to easily detect, with the detection function 188, whether or not the puncture needle 30 is present in the ultrasound image based on the first echo signal, which is a desired cross-sectional image.

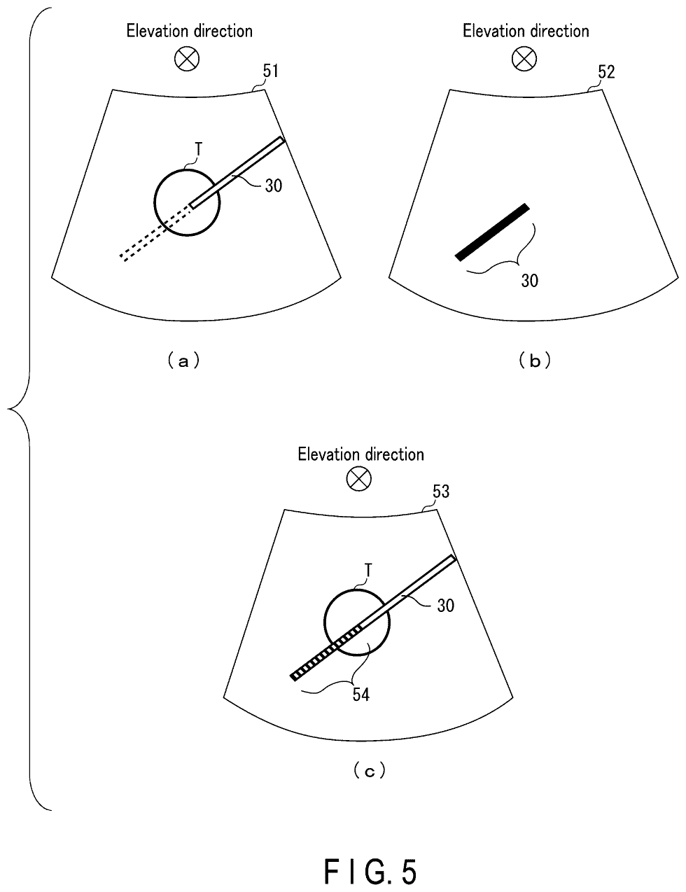

Next, a display example of an ultrasound image and a superimposition image according to the first generation example will be described with reference to FIG. 5.

FIG. 5(a) shows an ultrasound image 51 based on the first ultrasound beam 301, with the puncture needle 30 inserted through a target T. As shown in FIG. 4, since the puncture needle 30 is present outside of the area of the first ultrasound beam 301, the tip of the puncture needle 30 appears to be located at the central part of the tumor on the ultrasound image 51; however, part of the puncture needle 30, including the tip, is actually present outside of the plane of the ultrasound image 51, as shown by the dashed lines.

FIG. 5(b) shows a differential image 52 based on a differential signal between the first ultrasound beam 301 and the second ultrasound beam 302. With the differential image 52, it is possible to detect the presence of the puncture needle 30 in the spatial area of the second ultrasound beam 302.

FIG. 5(c) shows a superimposition image 53 in which the differential image 52 is superimposed on the ultrasound image 51. On the superimposition image 53, a portion of the puncture needle 30 depicted in the differential image 52 is displayed in a display color different from that of the puncture needle 30 depicted in the ultrasound image 51 and with a different pattern 54 in the example of FIG. 5. With the superimposition image 53 in which the portion of the puncture needle 30 that is present outside of the plane of the ultrasound image 51 is displayed with the different pattern 54, it is possible to easily grasp the presence of the puncture needle and the tip of the puncture needle outside of the plane of the ultrasound image 51.

The display form of the portion of the puncture needle 30 depicted in the differential image 52 is not limited to varying the display color and the hatching pattern, and other display forms may be adopted, such as blinking, boldfacing, etc. That is, any display form may be adopted as long as the portion of the puncture needle 30 depicted in the differential image 52 is distinguishably displayed on the superimposition image 53.

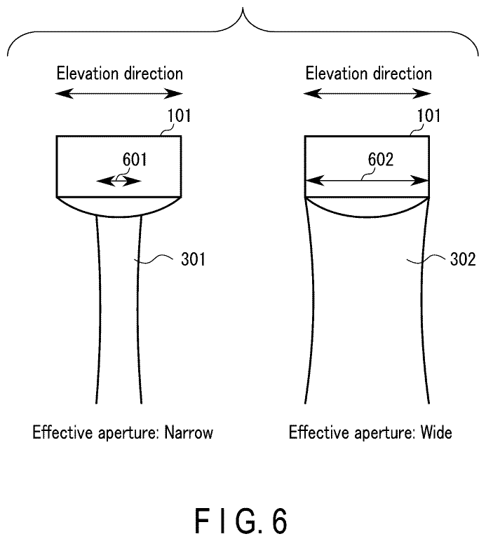

Next, a second generation example of an ultrasound beam according to the first embodiment will be described with reference to FIG. 6.

In the second generation example shown in FIG. 6, ultrasound beams have different apertures. The first ultrasound beam is set to have an effective aperture 601 that is narrow in the elevation direction of the ultrasound beam, and acquires a first echo signal. The second ultrasound beam is set to have an effective aperture 602 that is wider than the effective aperture 601 of the first ultrasound beam, and acquires a second echo signal.

In this manner, by varying the effective aperture of the ultrasound beam, it is possible to acquire echo signals from different areas. It is thus possible, with the second echo signal, to acquire a signal from outside of the area of the first echo signal, similarly to the first generation example.

According to the first embodiment described above, it is possible, with the acquisition function, to acquire a first echo signal and a second echo signal based on two ultrasound beams covering areas that overlap each other at least partially and differing from each other in the elevation direction. With the difference calculating function, a differential signal between the first echo signal and the second echo signal is calculated. With the display control function, a differential image based on the differential signal is displayed or superimposed on a desired ultrasound image.

It is thereby possible to grasp, based on whether or not a detection target is present in the differential image, whether or not the detection target is present outside of the plane.

### Second Embodiment

The second embodiment differs from the first embodiment in that a shift amount and a shift direction of a detection target are detected from a desired cross-sectional image by acquiring three ultrasound beams with different acquisition areas.

The configuration of an ultrasound diagnosis apparatus 1 according to a second embodiment is similar to that of the ultrasound diagnosis apparatus 1 according to the first embodiment.

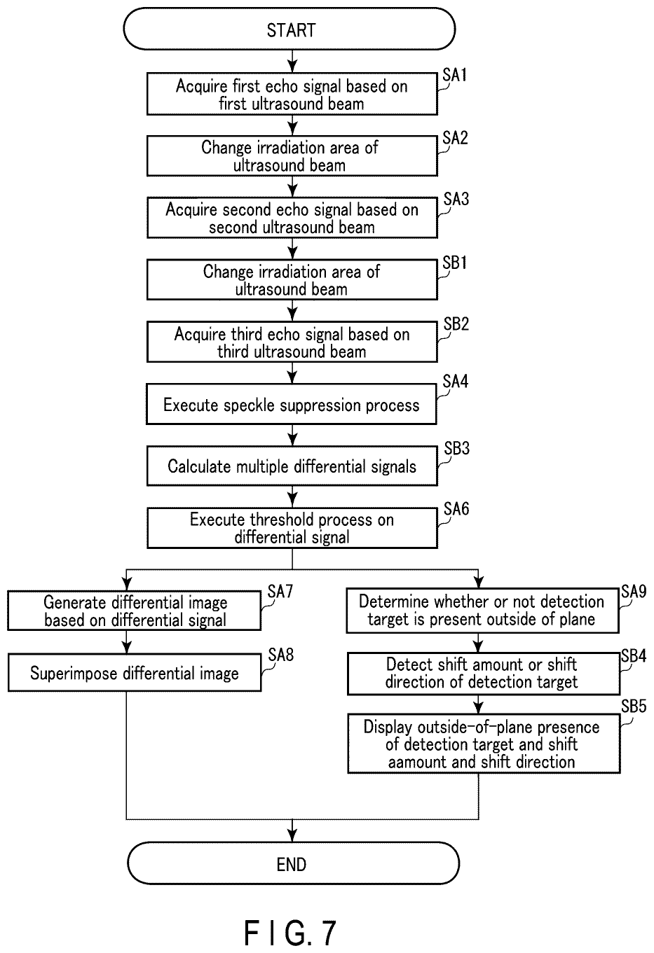

Next, an operation example of the ultrasound diagnosis apparatus according to the second embodiment will be described with reference to the flowchart in FIG. 7.

The processing at steps SA1 to SA3, SA4, and SA6 to SA9 is similar to that at the first embodiment.

At step SB1, the processing circuitry 180 sets, with the acquisition function 184, a beam irradiation area to be different from irradiation areas of the first and second ultrasound beams in an elevation direction.

At step SB2, with the acquisition function 184, the processing circuitry 180 acquires a third echo signal based on a third ultrasound beam, in accordance with the condition changed at step SB1. It is assumed that the third ultrasound beam covers an area encompassing the areas of the first and second ultrasound beams, or an area oriented oppositely to the area of the second ultrasound beam with reference to the area of the first ultrasound beam in the elevation direction.

At step SB3, the processing circuitry 180 calculates, with the difference calculating function 186, a difference between the first echo signal and the second echo signal subjected to the speckle suppression process, and calculates a first differential signal. The processing circuitry 180 further calculates a difference between the third echo signal and the first echo signal or the second echo signal, and calculates a second differential signal.

At step SB4, the processing circuitry 180 detects, with the detection function 188, a shift amount and a shift direction of the detection target. Specifically, in the case where, for example, the area of the third ultrasound beam encompasses the first ultrasound beam and the second ultrasound beam, if a comparison between the first differential signal and the second differential signal shows that the detection target is included in the first differential signal but not in the second differential signal, it is possible to determine that a detection target is not present in the area of the ultrasound beam corresponding to the third echo signal. It is thereby possible to detect a shift amount indicating a degree by which the detection target is shifted outside of the plane of the ultrasound image.

In the case where the area of the third ultrasound beam is oriented oppositely to the area of the second ultrasound beam with reference to the area of the first ultrasound beam in the elevation direction, by comparing the first differential signal and the second differential signal, it is possible to determine that the detection target is present in the direction of the second ultrasound beam or the direction of the third ultrasound beam, under the differential signal in which the detection target is included. It is thereby possible to detect a shift direction representing a direction in which the detection target is shifted outside of the plane of the ultrasound image.

At step SB5, the processing circuitry 180 displays, with the display control function 187, information as to whether or not the detection target is present outside of the plane, and displays, if the detection target is present outside of the plane, the shift amount or the shift direction on the screen. Such information may be displayed together with the superimposition image generated at step SA8.

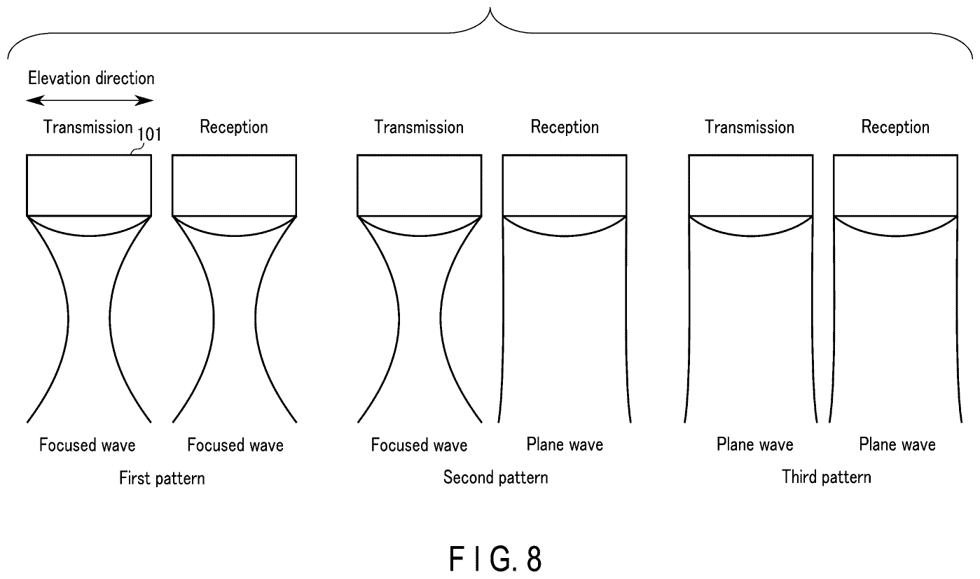

Next, a third generation example of an ultrasound beam in the case of detecting a shift amount of a detection target will be described with reference to FIG. 8.

In the third generation example shown in FIG. 8, three types of echo signals with different beam shapes in the elevation direction are obtained by changing the shape of the ultrasound beam between transmission and reception.

In a first pattern, the first ultrasound beam for acquiring a first echo signal is a focused wave with the beam focused at a particular depth, both in transmission and reception.

In a second pattern, the second ultrasound beam for acquiring a second echo signal is a focused beam in transmission, but is, in reception, a plane wave with the beam defocused.

In a third pattern, the third ultrasound beam for acquiring a third echo signal is a plane wave, both in transmission and reception. The thickness of the ultrasound beam in the elevation direction increases in the order of the first pattern, the second pattern, and the third pattern.

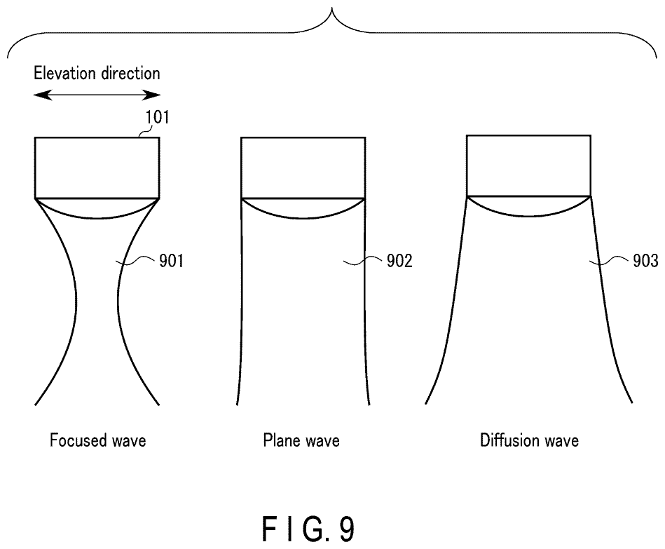

Next, a fourth generation example of an ultrasound beam, which is another example of detecting a shift amount of a detection target, will be described with reference to FIG. 9.

In the fourth generation example shown in FIG. 9, the shape of the ultrasound beam is changed similarly to FIG. 8, and three types of echo signals with different beam shapes in the elevation direction are obtained. In this example, the beam shape is varied as shown in FIG. 8, without distinction between transmission and reception.

The first ultrasound beam 901 for acquiring the first echo signal is a focused wave. The second ultrasound beam 902 for acquiring the second echo signal is a plane wave. The third ultrasound beam 903 for acquiring the third echo signal is a diffusion wave. The thickness of the ultrasound beam in the elevation direction increases in the order of the first ultrasound beam 901, the second ultrasound beam 902, and the third ultrasound beam 903.

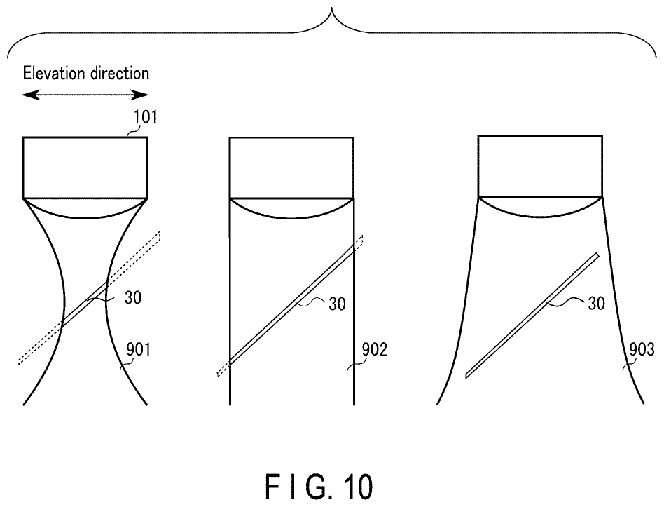

Next, beam setting for the detection target according to the fourth generation example will be described with reference to FIG. 10.

FIG. 10 shows an example in which an echo signal is acquired by each of the first ultrasound beam 901, the second ultrasound beam 902, and the third ultrasound beam 903 shown in FIG. 9, assuming depiction of the puncture needle 30. The portion in which the puncture needle 30 is depicted is shown by the solid lines, and the portion in which the puncture needle 30 is not depicted is shown by the dashed lines, similarly to the case of FIG. 4.

It is assumed that the puncture needle 30 is present in the area of each of the first ultrasound beam 901, the second ultrasound beam 902, and the third ultrasound beam 903 as shown in FIG. 10.

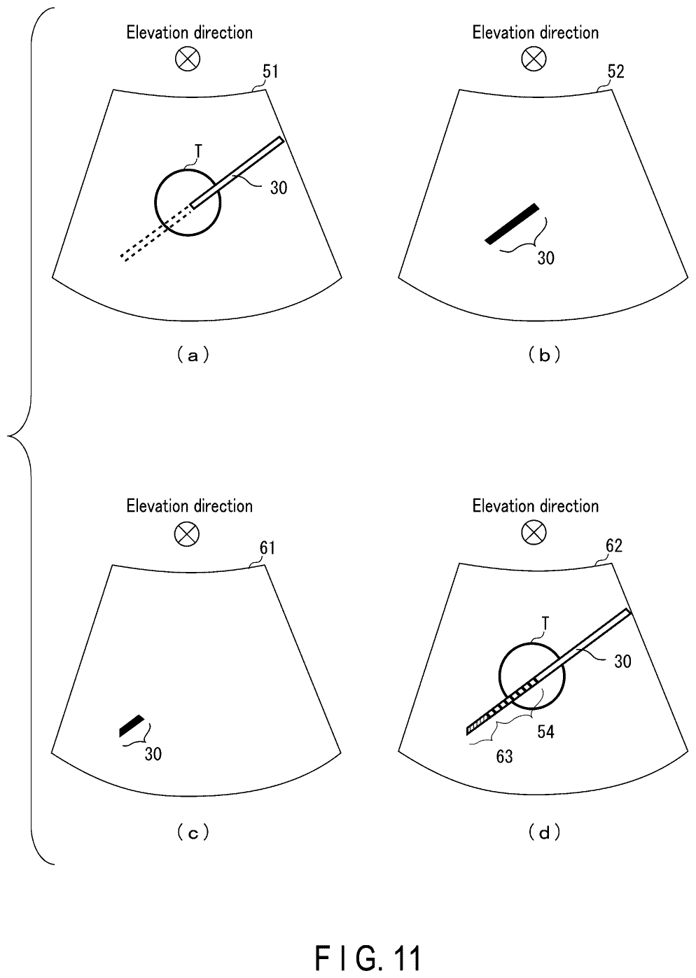

Next, a display example of an ultrasound image and a superimposition image according to the fourth generation example will be described with reference to FIG. 11.

FIG. 11(a) shows an ultrasound image 51 based on the first ultrasound beam 901, similarly to FIG. 5(a).

FIG. 11(b) shows a differential image 52 based on a first differential signal between the first ultrasound beam 901 and the second ultrasound beam 902. With the puncture needle 30 depicted in the differential image 52, it can be seen that the puncture needle 30 is present in the area of the second ultrasound beam 902.

FIG. 11(c) shows a differential image 61 based on a second differential signal between the second ultrasound beam 902 and the third ultrasound beam 903. With the puncture needle 30 depicted in the differential image 61, it is possible to detect the presence of the puncture needle 30 in the area of the third ultrasound beam 903.

FIG. 11(d) shows a superimposition image 62 in which the differential image 52 and the differential image 61 are superimposed on the ultrasound image 51. On the superimposition image 62, a portion of the puncture needle 30 depicted in the differential image 52 and a portion of the puncture needle 30 depicted in the differential image 61 are displayed with different patterns (a pattern 54 and a pattern 63). Specifically, a portion of the puncture needle 30 depicted in the differential image 52 is displayed with a pattern 54, and a portion of the puncture needle 30 depicted in the differential image 61 is displayed with a pattern 63. Note that, the processing circuitry 180 may be configured to calculate, with the detection function 188, the length of the puncture needle 30 shown with the pattern 54 and the pattern 63, and display, together with the superimposition image 62, the length information of an out-of-plane portion of the puncture needle 30 as a shift amount.

A case is assumed, for example, where the puncture needle 30 is present in the differential image 52 based on the first differential signal between the second echo signal and the first echo signal, but is not present in the differential image 61 based on the second differential signal between the third echo signal and the second echo signal. In this case, since the puncture needle 30 is present in the area of the second ultrasound beam 902 but is not present in the area of the third ultrasound beam 903, it can also be determined that the tip of the puncture needle 30 is present in the area of the second ultrasound beam 902. A similar process can be performed by using, as the second differential signal, a differential signal between the third echo signal and the first echo signal instead of the differential signal between the third echo signal and the second echo signal, and detecting whether or not the puncture needle 30 is present in the differential image 61 based on the second differential signal.

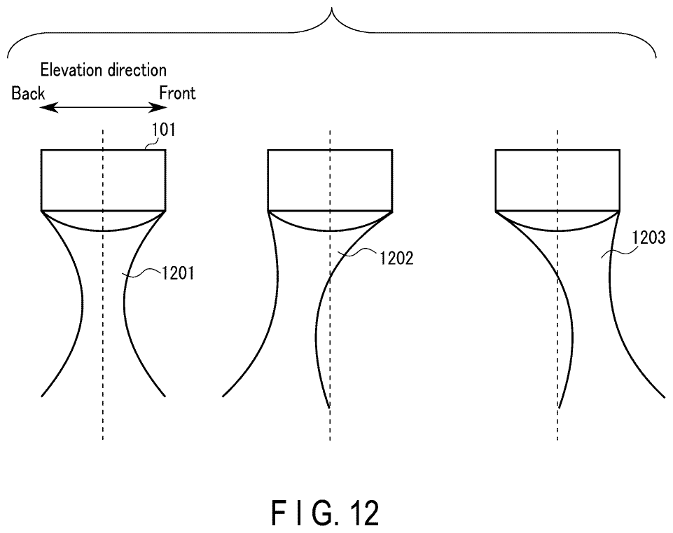

Next, a fifth generation example of an ultrasound beam in the case of detecting a shift direction of a detection target will be described with reference to FIG. 12.

The fifth generation example differs from the other generation examples in that data is acquired by shifting the delay time (delay pattern) among the plurality of elements of the ultrasound probe 101, thus tilting the beams in the elevation direction. Specifically, a first ultrasound beam 1201 is formed by focusing the beam as in a normal focused wave. The second ultrasound beam 1202 is formed by controlling the delay pattern of each element of the ultrasound probe 101 in such a manner that the beam has a directivity oriented toward a back side in the elevation direction. The third ultrasound beam 1203 is formed by controlling the delay pattern of each element of the ultrasound probe 101 in such a manner that the beam has a directivity oriented oppositely to the second ultrasound beam 1202, namely, toward a front side in the elevation direction with reference to the first ultrasound beam 1201.

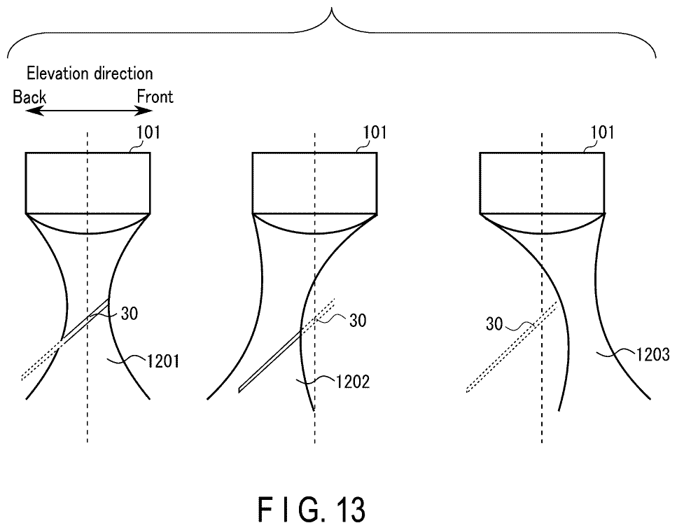

Next, beam setting for the detection target according to the fifth generation example will be described with reference to FIG. 13.

FIG. 13 shows an example in which an echo signal is acquired by each of the first ultrasound beam 1201, the second ultrasound beam 1202, and the third ultrasound beam 1203 shown in FIG. 12, assuming depiction of the puncture needle 30. The portion in which the puncture needle 30 is depicted is shown by the solid lines, and the portion in which the puncture needle 30 is not depicted is shown by the dashed lines, similarly to the case of FIG. 4.

As shown in FIG. 13, the puncture needle 30 is present in the area of each of the first ultrasound beam 1201 and the second ultrasound beam 1202. Furthermore, a case is assumed where the tip of the puncture needle 30 is located in the second ultrasound beam 1202.

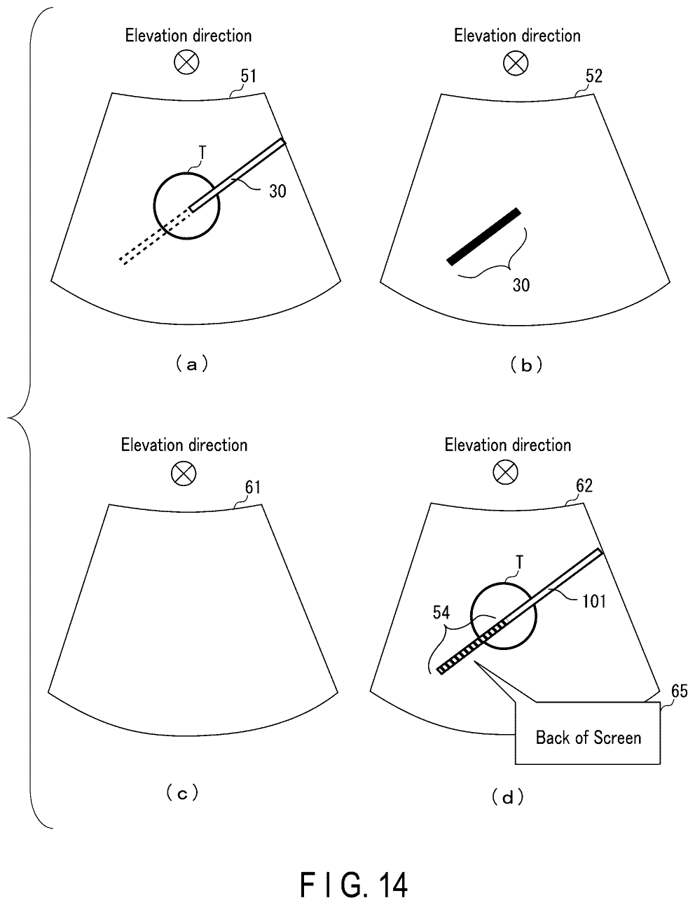

Next, a display example of an ultrasound image and a superimposition image according to the fifth generation example will be described with reference to FIG. 14.

FIG. 14(a) shows an ultrasound image 51 based on the first ultrasound beam 1201, similarly to FIG. 11(a).

FIG. 14(b) shows a differential image 52 based on a first differential signal between the first ultrasound beam 1201 and the second ultrasound beam 1202. With the puncture needle 30 depicted in the differential image 52, it can be seen that the puncture needle 30 is present in the area of the second ultrasound beam 1202.

FIG. 14(c) shows a differential image 61 based on a second differential signal between the second ultrasound beam 1202 and the third ultrasound beam 1203. In the differential image 61, the puncture needle 30 is not depicted. It can thus be seen that the puncture needle 30 is not present in the area of the third ultrasound beam 1203.

FIG. 14(d) shows a superimposition image 62 in which the differential image 52 and the differential image 61 are superimposed on the ultrasound image 51. On the superimposition image 62, a portion of the puncture needle 30 depicted in the differential image 52 is displayed with a different pattern 54 so as to be distinguishable from a portion of the puncture needle 30 depicted in the ultrasound image 51. Since it can be easily seen from the superimposition image 62 that the puncture needle 30 is present in the area of the second ultrasound beam 1202, it is possible to easily grasp the shift direction of the puncture needle 30, that is, whether the puncture needle 30 is present on the back side or the front side in the elevation direction, with reference to the ultrasound image 51.

A message 65 such as “Back of screen” and “Front of screen” may be displayed in a speech bubble, etc. from the pattern 54, as shown in FIG. 14(d). In addition, the display color may be fixed according to the shift direction to allow the user to recognize the shift direction. For example, a portion of the puncture needle 30 shifted to the back of the screen (in other words, the back of the ultrasound image 51) may be displayed in blue, and a portion of the puncture needle 30 shifted to the front of the screen (in other words, the front of the ultrasound image 51) may be displayed in red. It is thereby possible to allow the user to visually recognize the shift direction of the puncture needle 30.

Note that, with the detection function 188, the processing circuitry 180 may calculate the length of the puncture needle 30 shown with the pattern 54 and display, together with the superimposition image 62 and the shift direction, the length information of an out-of-plane portion of the puncture needle 30 as a shift amount.

In the fourth and fifth generation examples of the ultrasound beam, an example has been shown in which echo signals are acquired by ultrasound beams covering three different areas; however, the configuration is not limited thereto, and four or more echo signals may be acquired and a shift amount and a shift direction of a detection target may be detected. However, considering that the greater the number of echo signals used, the lower the frame rate becomes, resulting in a lower real-time performance, it suffices that the number of ultrasound beams used for processing be set according to the frame rate desired by the user.

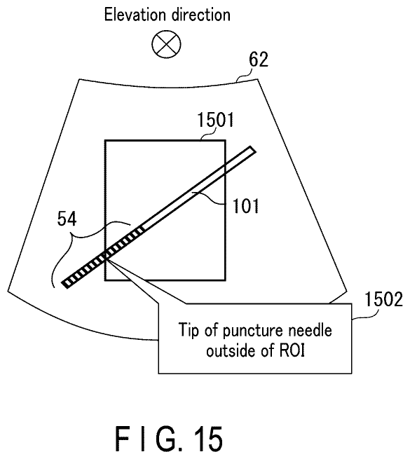

In the above-described example, a case is assumed where information as to whether or not the detection target is present outside of the plane of the ultrasound image is presented; however, information as to whether or not the detection target is within an ROI may be presented. A display example of an ultrasound image and a superimposition image in a case where an ROI is a target will be described with reference to FIG. 15.

FIG. 15 shows an example in which a superimposition image 62 shown in FIG. 14 is assumed, and an ROI 1501 is set in the ultrasound image 51. Upon detection of the puncture needle 30 outside of the plane of the ultrasound image 51, a message 1502 indicating, for example, “Tip of puncture needle outside of ROI” may be displayed, as shown in FIG. 15.

According to the second embodiment described above, it is possible, with the acquisition function, to acquire a first echo signal, a second echo signal, and a third echo signal based on three ultrasound beams covering areas that overlap one another at least partially and differing from each other at least in the elevation direction. With the difference calculating function, a first differential signal between the first echo signal and the second echo signal and a second differential signal between the third echo signal and the second echo signal or the first echo signal is calculated. With the display control function, a differential image based on the first and second differential signals is displayed or superimposed on a desired ultrasound image.

It is thereby possible to grasp, based on whether or not a detection target is present in the differential image, whether or not the detection target is present outside of the plane, and to detect a deviation amount and a deviation direction of the detection target, thus providing efficient information to the user.

The term “processor” used in the above explanation refers to, for example, a central processing unit (CPU), a graphics processing unit (GPU), or circuitry such as an application-specific integrated circuit (ASIC) or a programmable logic device (e.g., a simple programmable logic device (SPLD), a complex programmable logic device (CPLD), or a field programmable gate array (FPGA)). If the processor is, for example, a CPU, the processor reads programs stored in storage circuitry and executes the programs to implement the corresponding functions. On the other hand, if the processor is, for example, an ASIC, the corresponding functions are directly incorporated as logic circuitry in the circuitry of the processor instead of the programs being saved in the storage circuitry. It is also to be noted that each of the processors of the present embodiment is not necessarily configured as a single circuit, and may be configured as a single processor incorporating a plurality of independent circuits realizing the respective functions. Moreover, a plurality of constituent elements shown in the drawings may be integrated into a single processor to implement the corresponding functions.

Furthermore, the functions according to each of the embodiments may be realized by installing the programs for executing the above-described processes onto a computer such as a workstation, and developing them on a memory. At this time, a program for allowing a computer to execute the above-described method may be stored and distributed in a storage medium such as a magnetic disk (e.g., a hard disk), an optical disk (e.g., a CD-ROM, a DVD), or a semiconductor memory.

According to at least one embodiment described above, it is possible to easily grasp whether or not a detection target is present outside of the plane.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

What is claimed is:

1. An ultrasound diagnosis apparatus comprising processing circuitry configured to:
acquire a first echo signal based on a first ultrasound beam and a second echo signal based on a second ultrasound beam, the second ultrasound beam covering an area at least partially overlapping, and differing in an elevation direction from, an area covered by the first ultrasound beam;
calculate a differential signal representing a difference between the first echo signal and the second echo signal; and
display a differential image based on the differential signal.

2. The ultrasound diagnosis apparatus according to claim 1, wherein the processing circuitry is configured to execute a threshold process on the differential signal to extract a signal value which is equal to or greater than a threshold value, and
display the differential image based on the extracted signal value.

3. The ultrasound diagnosis apparatus according to claim 1, wherein the processing circuitry is further configured to detect whether or not a detection target is depicted in the differential image.

4. The ultrasound diagnosis apparatus according to claim 3, wherein the processing circuitry is configured to determine, if the detection target is depicted in the differential image, that at least a portion of the detection target is located outside of a plane of a desired cross-sectional image.

5. The ultrasound diagnosis apparatus according to claim 1, wherein the processing circuitry is further configured to:
execute a speckle suppression process on the first echo signal and the second echo signal; and
generate the differential signal based on the first echo signal and the second echo signal on which the speckle suppression process has been executed.

6. The ultrasound diagnosis apparatus according to claim 1, wherein the first ultrasound beam and the second ultrasound beam are ultrasound beams differing in frequency.

7. The ultrasound diagnosis apparatus according to claim 1, wherein the first ultrasound beam and the second ultrasound beam are ultrasound beams transmitted or received with different apertures in the elevation direction.

8. The ultrasound diagnosis apparatus according to claim 1, wherein the first ultrasound beam and the second ultrasound beam are formed to cover different areas by controlling a delay time of a signal in the elevation direction.

9. The ultrasound diagnosis apparatus according to claim 1, wherein the processing circuitry is configured to:
acquire a third echo signal based on a third ultrasound beam having a beam shape encompassing the areas of the first ultrasound beam and the second ultrasound beam;
calculate a first differential signal representing a difference between the first echo signal and the second echo signal and a second differential signal representing a difference between the third echo signal and the first echo signal or the second echo signal; and
detect, using the first differential signal and the second differential signal, a shift amount indicating a degree by which at least a portion of a detection target is shifted outside of a plane of a desired cross-sectional image.

10. The ultrasound diagnosis apparatus according to claim 1, wherein the processing circuitry is configured to:
acquire a third echo signal based on a third ultrasound beam, the third ultrasound beam covering an area at least partially overlapping the area of the first ultrasound beam and oriented oppositely to the area of the second ultrasound beam with reference to the area of the first ultrasound beam in the elevation direction;
calculate a first differential signal representing a difference between the first echo signal and the second echo signal and a second differential signal representing a difference between the third echo signal and the first echo signal or the second echo signal, and
detect, using the first differential signal and the second differential signal, a shift direction representing a direction in which at least a portion of a detection target is shifted in the elevation direction.

11. The ultrasound diagnosis apparatus according to claim 1, wherein the processing circuitry is configured to superimpose the differential image on an ultrasound image based on the first echo signal, in a display color different from the ultrasound image, and display a superimposed image.

12. An ultrasound diagnosis method, comprising:
acquiring a first echo signal based on a first ultrasound beam and a second echo signal based on a second ultrasound beam, the second ultrasound beam covering an area at least partially overlapping, and differing in an elevation direction from, an area covered by the first ultrasound beam;
calculating a differential signal representing a difference between the first echo signal and the second echo signal; and
displaying a differential image based on the differential signal.

13. A non-transitory computer readable storage medium storing a program that causes processing circuitry to:
acquire a first echo signal based on a first ultrasound beam and a second echo signal based on a second ultrasound beam, the second ultrasound beam covering an area at least partially overlapping, and differing in an elevation direction from, an area covered by the first ultrasound beam;
calculate a differential signal representing a difference between the first echo signal and the second echo signal; and
display a differential image based on the differential signal.
